(19) United States
(12) Patent Application Publication
(10) Pub. No.: US 2024/0415538 A1
(43) Pub. Date: Dec. 19, 2024

(54) FLEXIBLE TIP ATTACHMENT FOR LAPAROSCOPIC SUCTION/IRRIGATION DEVICE

(71) Applicant: Platform Innovations, Inc., Naples, FL (US)

(72) Inventor: Robert F. Novell, Colorado Springs, CO (US)

(73) Assignee: Platform Innovation, Inc., Naples, FL (US)

(21) Appl. No.: 18/747,815

(22) Filed: Jun. 19, 2024

Related U.S. Application Data
(60) Provisional application No. 63/508,977, filed on Jun. 19, 2023.

(51) Int. Cl. A61B 17/34; A61B 17/00; A61B 90/00

(52) U.S. Cl. CPC A61B 17/3421; A61B 17/00234; A61B 90/39; A61B 2017/00477; A61B 2017/00561; A61B 2017/00862; A61B 2017/00929; A61B 2017/3437; A61B 2090/3937; A61B 2217/005; A61B 2217/007

(57) ABSTRACT

A device for laparoscopic surgery comprising a tip having a main port at a distal end, a proximal port at a proximal end, a lumen extending from the main port to the proximal port, a flexible and non-conductive wall, and an attachment area extending from the proximal end toward the distal end, wherein the wall has a shape and the tip is removably attached to the rigid cannula of a laparoscopic device for suction or irrigation of a surgical site. Another embodiment includes a system for laparoscopic surgery comprising a tip having a main port, a proximal port, a lumen, and a flexible wall having a shape for improving access to a surgical site, wherein the tip is removably attached to the rigid cannula of a laparoscopic device. Another embodiment includes a method for performing laparoscopic surgery comprising removably attaching a flexible tip to the cannula of a laparoscopic device.

### CROSS-REFERENCE TO RELATED APPLICATIONS

This application claims priority to U.S. Provisional Patent Application No. 63/508,977, filed Jun. 19, 2023, and has specification that builds upon U.S. patent application Ser. No. 16/388,244, filed Apr. 18, 2019, which are hereby incorporated by reference herein in their entirety.

### BACKGROUND OF THE INVENTION

### Field of the Invention

Embodiments of the present invention relate generally to devices and systems comprising surgical devices and more particularly to attachments for a suction and/or irrigation device used in laparoscopy procedures.

### Relevant Background

Laparoscopic surgery is minimally invasive surgery, sometimes referred to as keyhole surgery, in which operations are performed through small incisions elsewhere on the body. During laparoscopic surgery, a long fiber optic cable system allows viewing of the surgical site by positioning the cable from a more distant, but easily accessed location. Laparoscopic surgery results in improved surgical outcomes by reducing surgical trauma for the patient.

Performing laparoscopic surgery requires that the surgical site be cleared of fluid and debris so that the surgeon may view the area being treated. Clearing the surgical site can be accomplished by introducing saline solution into the area and then removing the saline and debris via suction. Suction devices for laparoscopic procedures typically consist of straight rigid tubes inserted into the treatment area to introduce or remove fluid. However, concavities of the surgery site leave pockets or areas of fluid that are not readily accessible by a straight tube. Such pockets of fluid are currently removed via sponges which, in turn, must also be removed. A need therefore exists to introduce a flexible tip attachment that can be removably fixed to the distal end of a rigid suction device so that the combined system can maneuver within a surgical treatment site to introduce and remove fluid to aid in the viewing of and treatment of the surgical site. These and other deficiencies of the prior art are addressed by one or more embodiments of the disclosed invention.

Additional advantages and novel features of this invention shall be set forth in part in the description that follows, and in part will become apparent to those skilled in the art upon examination of the following specification or may be learned by the practice of the invention.

### BRIEF DESCRIPTION OF THE DRAWINGS

Features and objects of the present invention and the manner of attaining them will become more apparent, and the invention itself will be best understood, by reference to the following description of one or more embodiments taken in conjunction with the accompanying drawings attached following this description.

FIGS. 1A, 1B, and 1C show a prior art laparoscopic suction/irrigation device having a rigid probe or cannula.

FIG. 2 shows a side view of a laparoscopic suction/irrigation device modified with a flexible tip attachment according to one embodiment of the disclosed invention, wherein the tip attachment has a straight shape configuration.

FIGS. 3A and 3B show a side view, and insets show a translucent view, of a flexible tip attachment fitted to a laparoscopic suction/irrigation device, according to embodiments of the disclosed invention.

FIGS. 4A, 4B, 4C, and 4D show a side view of flexible tip attachments having various shape configurations, according to embodiments of the disclosed invention.

FIGS. 5A, 5B, 5C, and 5D show a side view of flexible tip attachments having various venting port configurations, according to embodiments of the disclosed invention.

FIGS. 6A, and 6B show a side view, and insets show a close-up view, of flexible tip attachments having various specialized terminal area configurations, according to embodiments of the disclosed invention.

FIGS. 7A, and 7B show a side view of flexible tip attachments having various fiducial marking configurations, according to embodiments of the disclosed invention.

FIG. 8 shows a side view of a laparoscopic suction/irrigation device modified with a flexible tip attachment according to embodiments of the disclosed invention, wherein the system includes a suction source and an irrigation source.

FIGS. 9A and 9B show a side view, and an inset shows a close-up bottom view, of a laparoscopic suction/irrigation device modified with a flexible tip attachment for dispensing medication according to embodiments of the disclosed invention.

FIGS. 10A, 10B, and 10C show a side view of a laparoscopic suction/irrigation device modified with interchangeable rigid cannulas with fixed flexible tip attachments having various shape configurations, according to embodiments of the disclosed invention.

FIG. 11 is a flowchart for one embodiment of a methodology for the use of a laparoscopic suction/irrigation device modified with a flexible tip attachment of the disclosed invention.

The Figures depict embodiments of the disclosed invention for purposes of illustration only. One skilled in the art will readily recognize from the following discussion that alternative embodiments of the structures and methods illustrated herein may be employed without departing from the principles of the invention described herein.

### DETAILED DESCRIPTION

The invention described herein are devices and systems comprising a flexible tip attachment for use with a laparoscopic irrigation and/or suction device. A rigid laparoscopic device fitted with the disclosed flexible tip can be positioned within a surgical site to provide discrete and localized fluid evacuation and irrigation. The disclosed invention introduces a flexible, shaped probe into a laparoscopic surgical environment allowing surgical personnel to quickly and efficiently evacuate and irrigate the surgical site.

Embodiments of the present invention are hereafter described in detail with reference to the accompanying Figures. Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention.

The following description with reference to the accompanying drawings is provided to assist in a comprehensive understanding of exemplary embodiments of the disclosed invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

The terms and words used in the following description and claims are not limited to the bibliographical meanings, but are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

The terminology used herein is for the purpose of describing particular embodiments only and is not intended to be limiting of the invention. As used herein, the singular forms “a,” “an” and “the” are intended to include the plural forms as well, unless the context clearly indicates otherwise. Thus, for example, reference to “a component surface” includes reference to one or more of such surfaces.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present), and B is false (or not present), A is false (or not present) and B is true (or present), and both A and B are true (or present).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be also understood that when an element is referred to as being “on,” “attached” to, “connected” to, “coupled” with, “contacting”, “mounted” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

### Laparoscopic Suction/Irrigation Device

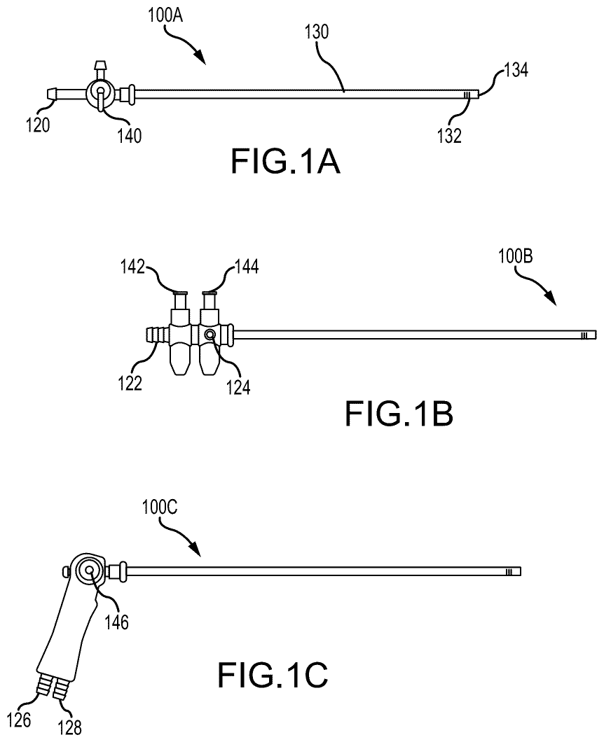

The disclosed flexible tip attachments are configured to be used with existing rigid laparoscopic suction and/or irrigation devices. When performing suction, the suction/irrigation device is used to remove fluid and debris from the surgical site, keeping the area clean and dry. When used for irrigation, the device flushes the surgical site with saline or another solution, allowing the surgeon to visualize the surgical field clearly. With reference to FIGS. 1A, 1B, and 1C, three representative devices are depicted. FIG. 1A depicts a reusable device 100A equipped with a single connection 120, for connecting either a suction or vacuum source or a fluid source via tubing (not shown). The device includes a rigid cannula 130 with venting ports 132, a main port or opening 134 at the distal end of the cannula, and an internal passageway or lumen for conveying fluid into or out of the body. A thumb-type actuator 140 is used to selectively turn the suction or irrigation on and off. FIG. 1B depicts a trumpet-style disposable device 100B having two connections: one for a suction source 122 and one for a fluid source 124. A suction actuator 142 allows the user to apply suction to the surgical site, while an irrigation actuator 144 allows the application of fluid to the site. FIG. 1C depicts a pistol-grip style reusable suction/irrigation device 100C. The pistol-grip style device also includes a suction connector 126 and an irrigation connector 128. Also included is an actuator 146 having a selectable off position for turning the device off, a suction position for applying suction to the surgical site, or an irrigation position for supplying fluid to the surgical site.

### Flexible Tip Attachment

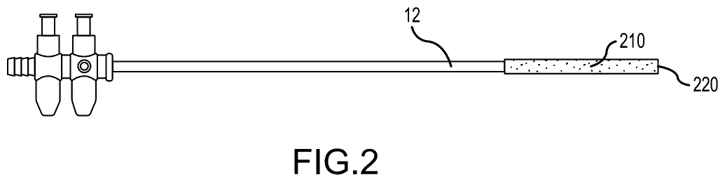

With reference to FIG. 2 is depicted a side view of a flexible tip attachment 210 that is removably attached as an accessory to a preexisting rigid laparoscopic suction and/or irrigation device, such as those depicted in FIGS. 1A to 1C referenced above. The flexible tip 210 is a hollow tube that is open at both ends and is attached to the distal end of a cannula 12 of the suction/irrigation device by an attachment means. The flexible tip has a main port 220 at its distal end for dispensing saline irrigation fluid into the surgical site, or for accepting fluid and debris from the surgical site. A lumen is created by and located within a flexible wall. The lumen extends the length of the tip attachment and serves a conduit for transporting fluid and or debris between the surgical site and the lumen of the rigid cannula 12. A proximal port or opening at the proximal end of the tip attachment allows fluidic communication between the cannula and the lumen.

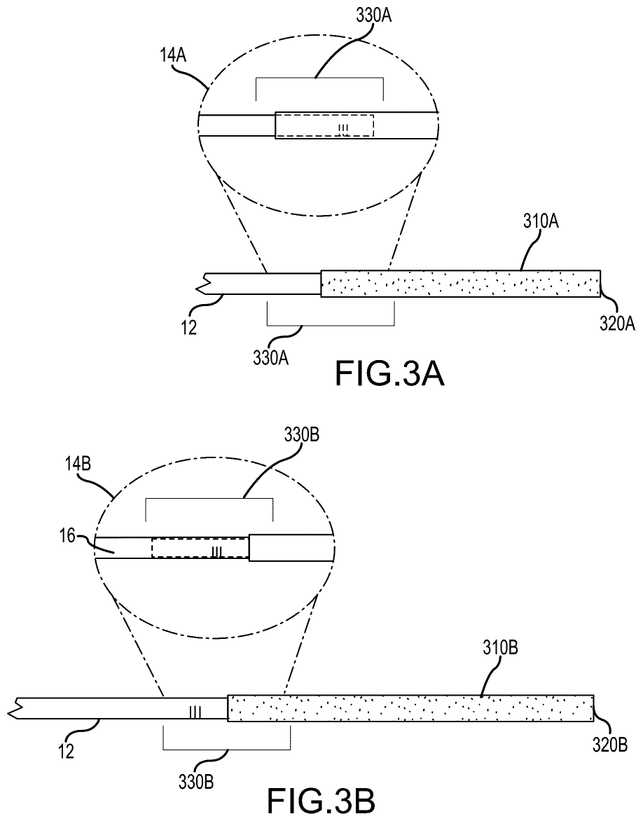

With reference to FIGS. 3A and 3B is depicted a close-up side view of a disclosed flexible tip attachment 310A that is removably attached to the cannula 12 of a laparoscopic suction/irrigation device. Insets 14A, 14B depict details of the attachment means by showing a translucent view of an attachment area 330A, 330B located at and extending from the proximal end of the tip attachment. In some embodiments, the attachment area 330A is configured to fit over the end of the laparoscopic device cannula 12. In such cases, the lumen of the tip attachment has an inner diameter sized to accommodate the outer diameter of the cannula, and the inner surface of the wall mechanically interacts with the outer surface of the cannula. In other embodiments, as shown in the inset 14B, the attachment area 330B is configured to fit through the main port of the cannula 12 and fit inside a portion of the cannula lumen 16. In such cases, the attachment area has an outer diameter sized to fit within the diameter of the cannula lumen, i.e., the inner diameter of the cannula. The outer surface of the wall in the attachment area mechanically interacts with the inner surface of the cannula. The flexible tip 310A, 310B is attached to the cannula 12 by a friction fit, temporary adhesive, or other suitable means, so that the tip will remain in place during surgery, but may be removed when required by the user. The attachment forms an air and fluid tight seal between the cannula and the tip attachment to facilitate extending suction and/or irrigation from the distal end of the cannula through the inner lumen, to the distal end of the tip attachment, and through the main port 320A, 320B.

The flexible tip 310A, 310B is shown in a linear or straight configuration, wherein the tip extends in line with the cannula 12. Different lengths are possible and contemplated for all flexible tip configurations, with a shorter version 310A and a longer version 310B shown. Flexible tip attachment sizes, lengths, and configurations may be chosen by the surgeon, depending on the needs of the surgery being performed.

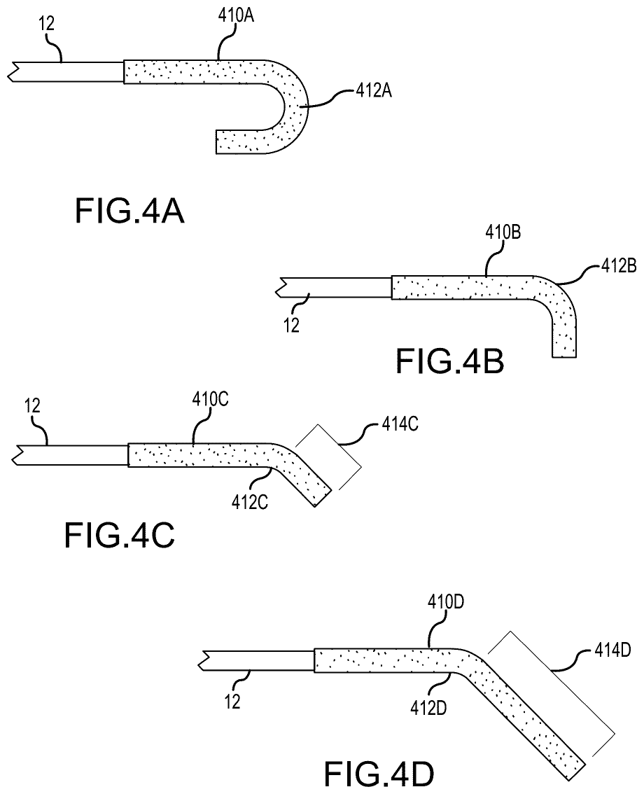

In addition to the straight configuration, the disclosed flexible tip attachment may have a preformed shape of any configuration of benefit. With reference to FIG. 4A, a flexible tip attachment 410A is removably secured to the distal end of a cannula 12, and its wall is shaped with a 180 Degree (°) curve 412A. FIG. 4B depicts a tip attachment 410B with its wall shaped with a 90° curve 412B. With reference to FIG. 4C, the tip attachment 410C has its wall shaped with a 45° curve or bend 412C, and has a terminal section or extension 414C extending the distal end of the tip attachment past the bend. With reference to FIG. 4D, the tip attachment 410D has a wall with a 45° bend 412D, and has a terminal section 414D that is longer than the extension 414C of the prior figure.

Multiple curve or bend angles are possible and contemplated, as are multiple extension lengths for each shape configuration, all to be determined based on the needs of the surgeon. For example, a straight tip attachment like that depicted in FIG. 2 might be most useful during surgery for blunt force or abrasion trauma. An attachment having a curve similar to that depicted in FIG. 4B with a 1-inch radius may be particularly adapted for use in general bowel surgery or removal of kidney tumors. Likewise, a similar curve shape with an extension may be suitable for accessing the posterior cul-de-sac area during gynecological procedures. A 45° bend with extension, such as is depicted in FIG. 4D, may be most useful during rectosigmoid surgeries. The 180° curve, such as shown in FIG. 4A, may be used to provide hook retraction.

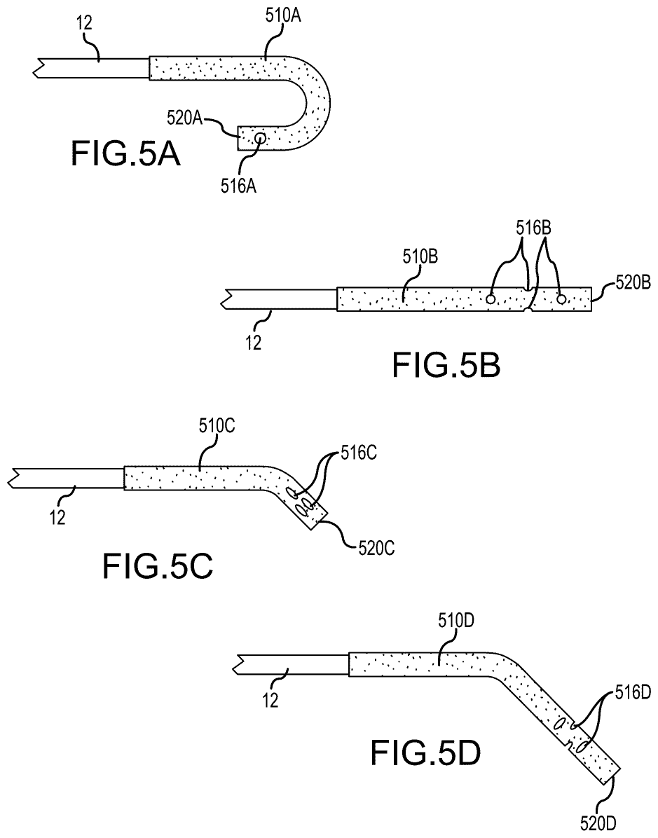

With reference to FIGS. 5A, 5B, 5C, and 5D, embodiments of the disclosed flexible tip attachment include venting ports or holes in the tip wall, which may assume various shapes and configurations, depending on the preferred use in surgery, or performance characteristics of a particular tip attachment shape. The venting ports are arranged in a terminal section of the tip, the terminal section being a portion of the length of the tip extending back from the distal end toward the proximal end. In other words, the venting ports are located closer to the distal end than to the proximal end. Venting ports facilitate fluid force transfer at the surgical site, allowing fluid and/or debris to be ejected from inside the lumen of the tip attachment.

A tip attachment having an open main port with vents, known as a Frazier or Yankauer tip, avoids tissue entrapment during suction by releasing the pressure differential created when the main port is blocked. During irrigation, the Frasier tip can disperse fluid more readily because of the additional avenues for dispersal. The number and size of venting ports is chosen to balance the increased dispersion and decreased tissue entrapment with the decrease in main port fluidic pressure that result from increasing the number or size of venting ports. A tip attachment with a closed main port with vents, known as a Poole or sump tip, allows the user to suction large amounts of fluid while decreasing the chance of the tip becoming blocked by anatomy or debris, e.g., bowel contents. During irrigation, the Poole tip disperses fluids widely because of the increased pressure supplied to the venting ports.

FIG. 5A depicts a flexible tip attachment 510A removably fixed to the distal end of a cannula 12, and having a 180° curve. The tip attachment 510A includes one or more venting ports 516A having a circular shape and located on a lateral side of the attachment. FIG. 5B depicts a tip attachment 510B having a plurality of venting ports 516B having a circular shape, and arranged on a lateral side, a dorsal side, and a ventral side of the tip attachment. FIG. 5C depicts a tip attachment 510C having one or more venting ports 516C, wherein the venting ports have an oval shape, and are arranged in a group on a lateral side of the tip attachment. FIG. 5D depicts a tip attachment 510D having a plurality of venting ports 516D having an oval shape, and arranged on a lateral side, a dorsal side, and a ventral side of the tip attachment. Venting ports may be located various distances from the main port 520A, 520B, 520C, 520D up to the distal end of the cannula 12, depending on the desired function.

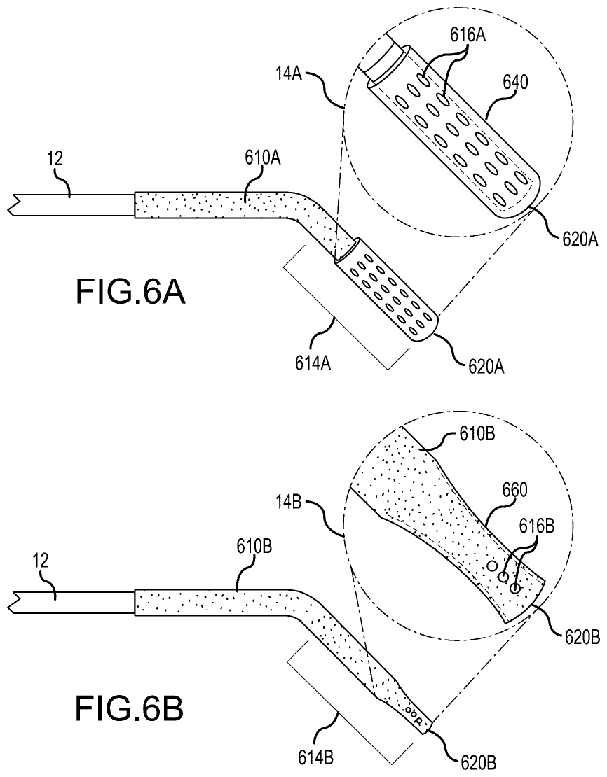

With reference to FIGS. 6A and 6B the disclosed flexible tip attachment may include a specialized terminal section 614A, 614B. While the drawings depict a tip attachment with a 45° bend, other shapes may have specialized terminal sections as required. The disclosed specialized terminal sections may include a preformed shape configuration, such as a Frasier Tip, Poole Tip, Sump Tip, or other suitable configuration. FIG. 6A depicts a flexible tip attachment 610A removably fixed to the distal end of a cannula 12, and having a specialized terminal section 614A known as a Poole Tip. An inset 14A shows the relevant area in detail. The terminal section includes a molded tip 640 that comprises the distal end of the tip attachment. The tip includes one or more venting ports 616A, here a regular pattern of vents is arrayed around the tip. The sleeve also includes a closed main port 620A. The tip 640 may be molded out of the tip attachment material or it may be attached. If attached, the tip material may be the same, or it may be made from other suitable material, and may be flexible or rigid, as required.

FIG. 6B depicts a flexible tip attachment 610B removably fixed to the distal end of a cannula 12, and having another embodiment of a specialized terminal section 614B. An inset 14B shows the relevant area in detail, depicting a translucent view of the terminal section. In this embodiment, the terminal section includes a flared tip 660 having a modified main port 620B. As depicted, the flared tip has a narrower cross-section than the remainder of the tip attachment 610B. The flared tip also includes one or more venting ports 616B located near the main port and aligned longitudinally centered on the lumen. The narrow cross section of the lumen in the flared tip increases fluid velocity given constant suction or irrigation pressure, allowing the tip attachment to meet different performance characteristics when so equipped. The flared tip 660 may be molded out of the tip attachment material, or the insert may be attached, and may be made of the same material, or other suitable material, and may be flexible or rigid, as required. Multiple specialized terminal area modifications are possible and contemplated.

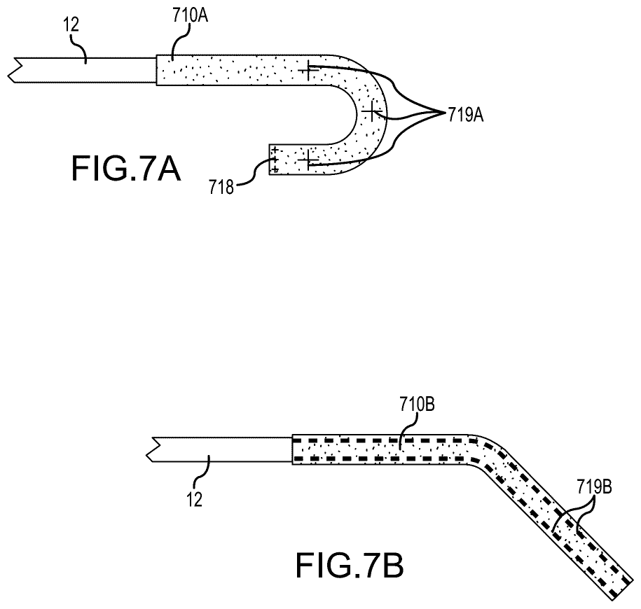

With reference to FIGS. 7A and 7B, the flexible tip attachment 710A, 710B may include fiducial markers to aid in its positioning. Some fiducial markers 718 may designate the distal end of the tip attachment, others may outline 719A, 719B the curve of the tip attachment to help orient the user, or other suitable arrangement. Fiducial markers may be incorporated or embedded in material comprising the tip attachment, or may be adhered to, printed or painted on the surface. Fiducial markers may include technology to improve visibility at the surgical site, such as reflectiveness or phosphorescence.

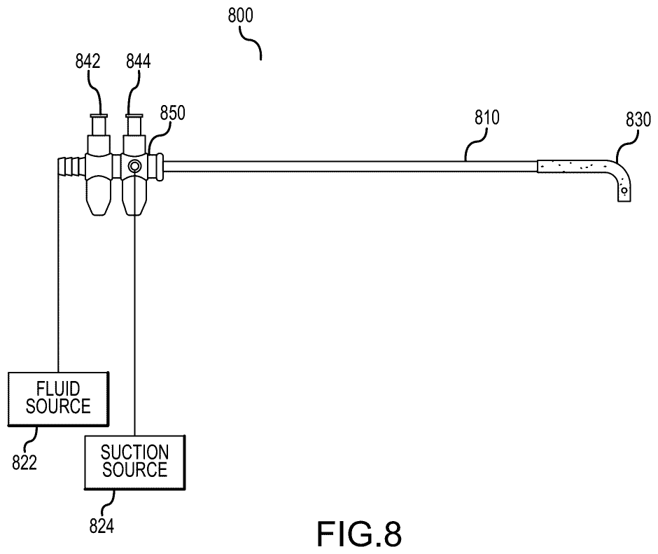

FIG. 8 shows a high-level view of a laparoscopic device 800 for use with the disclosed invention. In use, the disclosed flexible tip attachment 830 is removably attached to a rigid cannula 810 of a laparoscopic device body 850. The combined system is then inserted into a patient's body through a laparoscopic trocar lumen, which has been inserted through a keyhole-type incision in the body. The trocar functions as a portal for the placement of laparoscopic instruments, such as graspers, scissors, staplers, etc., including the laparoscopic suction/irrigation device 800. The flexible tip 830 will deflect and fold along the length of the cannula 810 until the system is though the trocar and into the body cavity. Then the flexible tip will return to its original functional position, which provides the benefit of the selected preformed shape.

The laparoscopic system can then perform the irrigation and/or suction tasks in the areas of the surgical site facilitated by the selected flexible tip shape. To perform evacuation of the surgical site, the surgeon would select a suction control 844 that activates a suction source 824 connected to the system through medical tubing (not shown). For irrigation of the site, the surgeon would select an irrigation control 842, which would activate a fluid source 822, also connected by medical tubing (not shown). As is well known and discussed herein, laparoscopic surgery requires removal of debris and fluid at the surgical site to aid in visualization. Conversely, hydro-dissection (using high pressure fluid as a cutting tool) can also be used as an alternative to traditional cutting tools. The system shown in FIG. 8 may also include regulators and hand/foot controls to assist in the use of the suction/irrigation device.

When removal of the system from the surgical site is desired, the flexible tip 830 will again deflect, this time to trail behind the cannula 810, if necessary, as the system is drawn back through the laparoscopic trocar and out of the body. The user may then remove the attached flexible tip and attach another tip having a different shape to facilitate access to a different area of the surgical site, or to perform some other surgical task.

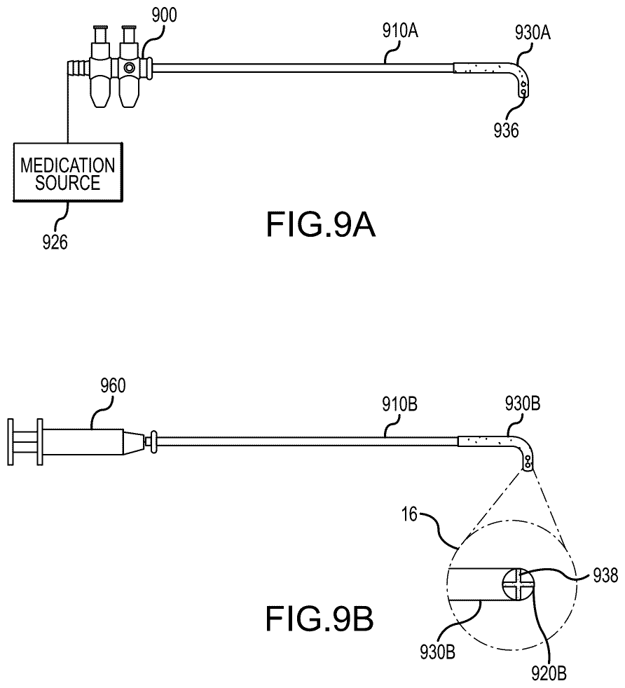

In some embodiments, the system shown in FIG. 8 may be used to deliver and direct medications to a surgical site. In particular, the tip attachment is configured to deliver medications not normally included in irrigation of a laparoscopic surgical site. For example, with reference to FIG. 9A, the rigid cannula 910A is fitted with a tip attachment 930A for medication delivery. The terminal area and main port of the tip attachment may have any configuration beneficial to the disbursement of medication, to include venting ports 936, and a modified main port. For example, with reference to FIG. 9B, inset 16 shows a close-up bottom view of the tip attachment 930B. The tip attachment is shown with an atomizer modification 938 to the main port 920B. The atomizer 938 improves the delivery of low viscosity medications. The cannula 910A and tip attachment 930A may be used with the laparoscopic device 900, or the cannula 910B and tip attachment 930B may be used independently. Medication is delivered via a medication source 926, such as a medication pump, medication delivery system, or syringe 960.

The curved flexible tip attachment enables the user to position the laparoscopic suction/irrigation device around anatomical structures and into concave areas. The tip attachment can also help deconflict with other laparoscopic instruments being used at the surgical site and improve visualization. Rather than displacing the laparoscopic instrument to allow a traditional suction/irrigation device into the surgical site, the disclosed invention allows a properly fitted suction/irrigation device to remain out of the way while the distal end of the flexible tip attachment reaches the remote area to evacuate or irrigate the site. Further, because the tip attachment is non-conductive, it can be used in proximity to laparoscopic tools that perform cauterization or dissection without the risk of electrical arcing. The flexible tip attachment thus allows other surgical tools to remain at the surgical site while the modified laparoscopic device restores visualization. Such flexibility and concurrent use of the modified suction/irrigation device allows the surgical team to work more efficiently.

For example, typically a surgeon would use laparoscopic devices at the surgical site, invariably producing fluids and debris that obscure the surgeon's visualization. To restore visualization, the surgeon would withdraw these tools to use a laparoscopic suction/irrigation device fitted with a rigid, often metallic, suction tip. The rigid nature of existing laparoscopic suction tips, however, often causes difficulty in reaching certain areas of the surgical site, resulting in ineffective evacuation of fluids and debris. Additionally, since they are often metallic, existing suction tips also carry a risk of monopolar electrical arcing when used with dissection tools, potentially resulting in patient injury.

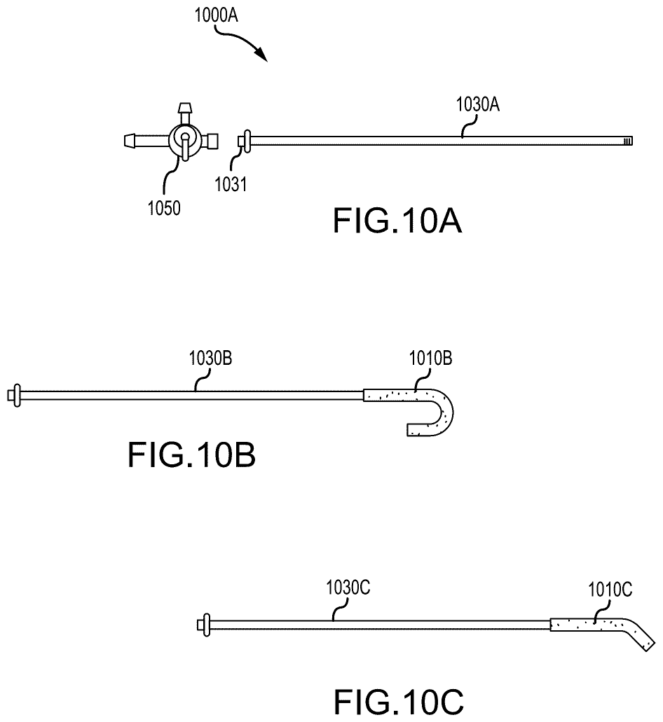

With reference to FIGS. 10A, 10B, and 10C, another embodiment of the disclosed invention is depicted. Such embodiment is suitable for use with a suction/irrigation laparoscopic device 1000A configured for use with removable rigid suction/irrigation attachments 1030A. Such attachments would have a connector 1031 at the proximal end to facilitate connection to the body section 1050 of the suction/irrigation device. With reference to FIGS. 10B, and 10C, the disclosed flexible tip attachment 1010B, 1010C is permanently fixed to the distal end of the attachments 1030B, 1030C. A laparoscopic suction/irrigation system of such embodiment would include multiple rigid attachments, each with a flexible tip attachment having a different preformed shape. A system user would select a rigid attachment 1030B having the required flexible tip shape, which could be used and then exchanged for a different attachment 1030C if desired.

In another embodiment, a flexible tip attachment is permanently bonded to a rigid attachment that is permanently incorporated into a laparoscopic device.

The flexible tip attachment may be made from medical grade tubing. Medical tubing is used for fluid management and drainage as well as with anesthesiology and respiratory equipment, IVs, catheters, peristaltic pumps, and biopharmaceutical laboratory equipment. There are a broad range of suitable materials used to construct medical tubing. As will be appreciated by one or reasonable skill in the relevant art, the type of material used in the flexible tip attachment determines important performance properties such as abrasion resistance, hardness, flexibility, and durability. Moreover, the pressure rating and vacuum rating of the flexible tip attachment is considered in selecting the grade and specification of the material used in its construction. Materials suitable for use in construction of the flexible tip attachment can include Ethylene Propylene (EP), Fluoro-elastomer (FKM), Isobutylene Isoprene Butyl (IIR), Isoprene (IR), Nitrile Rubber (NBR), Polychloroprene (CR), Polyurethane (PU), Silicone Rubber, Styrene-butadiene (SBR) and the like. Alternatively, the flexible tip attachment may be made of any suitable medical grade, sterilizable, and flexible material, e.g., silicone, nylon, polyurethane, polyurethane terephthalate, latex, elastomers, etc.

The distal end of the flexible tip attachment can achieve 180° deflection, 90° deflection, 450 deflection, or other required angle with respect to the distal end of the rigid cannula of a traditional laparoscopic suction/irrigation device. Curves may be shaped as a tight curve, a long curve, or a slight curve with long extension. The tip attachment may also have a taper wherein the diameter gradually decreases toward the main port. The curved configuration of the flexible tip attachment can be achieved by various techniques known in the art. For example, the shape of the tip attachment may be a pre-formed shape, or may be formed by using a mold, such as through an injection molding process. Alternatively, temperature curing techniques, such as thermoforming or vacuum forming, or any other suitable manufacturing process may be used to create the required curve.

Another feature of the flexible tip attachment is its insulating characteristics. During laparoscopic surgery, the presence of surgical devices using monopolar electricity can result in electrical arcs discharged at the surgical site. If the surgeon is controlling active bleeding or performing dissection with electrocautery, the flexible tip attachment can be used to provide better visualization, better outcomes, and create a safer environment since it can be used closer to electrocautery without causing arcing. Cauterization devices use electrical arc technology to fuse tissue and control hemorrhaging at the surgical site. Such electrical discharges, when uncontrolled, can be harmful. Without the disclosed tip attachment in place, the traditional metallic suction/irrigation devices can cause arcing to occur when used in proximity to electrocautery devices, which may injure adjacent tissue. The flexible tip attachment insulates metallic suction/irrigation devices, substantially reducing or even eliminating any arcing risk.

Another feature of the flexible tip attachment is its ability to protect delicate tissue from abrasion or penetrating injury caused by the rigid metal tips currently in use.

### METHODOLOGY FOR USE

In another embodiment of the present invention, a method for localized fluid injection, or fluid and debris removal, during laparoscopic surgery using the disclosed invention includes positioning the distal end of the tip attachment proximate to a surgical site. As described above, a flexible tip attachment of the required shape is removably attached to the distal end of the cannula of a laparoscopic suction/irrigation device. The tip attachment shape is selected based on the needs of the surgeon to improve visualization as required by the shape of the surgical cavity, the presence of other laparoscopic instruments, or the location of needed suction or irrigation. The modified suction/irrigation device thus includes an elongated substantially rigid cannula having a lumen extending from its distal end to its proximal end. The device further includes the flexible tip attachment having a main port and a lumen extending therethrough. A portion of the tip attachment overlaps a portion of the rigid cannula and a removable seal is created. The shape of the modified rigid cannula with tip attachment is helpful to reach concavities at the surgical site. Upon applying a fluid force to the proximal end of the rigid cannula, suction/irrigation device removes fluid from the surgical site (by application of a suction force) and/or injects fluid to the surgical site (upon application of an irrigation force).

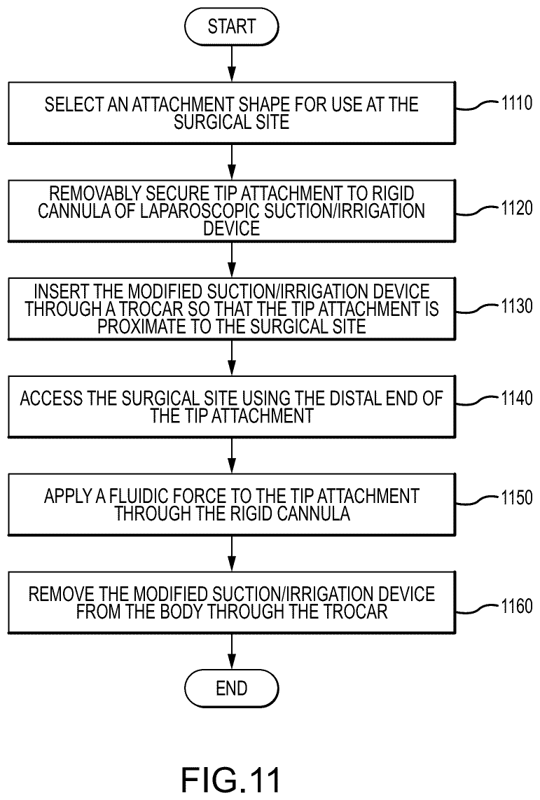

FIG. 11 presents a flowchart depicting a method for using a modified suction/irrigation device in laparoscopic surgery consistent with the disclosed invention. In practice, the flexible suction/irrigation system is used in conjunction with other laparoscopic tools which are all introduced proximate to the surgical site via a trocar. Such surgical use methods are well known and the specifics of their application within the context of the disclosed invention will be readily apparent to one of ordinary skill in the relevant art in light of this specification. Prior to introduction of the system into the trocar, a flexible tip attachment is selected 1110 as needed to access the surgical site. Then the attachment is removably attached to the distal tip of a rigid cannula 1120 as shown in the prior figures. The tip of the laparoscopic suction/irrigation device is then inserted through the lumen of a laparoscopic surgical trocar proximal to a surgical site 1130, for example, the abdomen of a patient undergoing laparoscopic surgery. The flexible tip attachment folds along the length of the rigid cannula while in the trocar and returns to its preformed shape once in the surgical cavity. The surgeon uses the suction/irrigation device to direct the distal end of the tip attachment to a position inside the patient where suction or irrigation needs to be applied 1140 to facilitate the laparoscopic procedure to be performed.

The flexible tip attachment is positioned around anatomical structures to access areas at the surgical site that would be otherwise unreachable with a straight rigid cannula to facilitate effective irrigation or fluid/debris evacuation. Once positioned, a fluidic force (suction or pressure) to the flexible tip attachment is applied via the rigid cannula 1150. Upon completion of the evacuation or irrigation, the fluid force is removed, and the device can then be repositioned or removed from the body 1160. Once outside the body, the user may remove the tip attachment and replace it with an attachment having a different shape to access other areas in the surgical site to perform dissection, retraction, suction, or irrigation. The process can be performed at any time as needed during the procedure to exploit the benefits of the different tip attachment shapes.

As will be understood by those familiar with the art, the disclosed invention may be embodied in other specific forms without departing from the spirit or essential characteristics thereof. Likewise, the particular naming and division of the modules, managers, functions, systems, layers, features, attributes, methodologies, and other aspects are not mandatory or significant, and the mechanisms that implement the invention or its features may have different names, divisions, and/or formats. Accordingly, the disclosure of the present invention is intended to be illustrative, but not limiting, of the scope of the invention.

This has been a description of the disclosed invention along with a preferred method of practicing the invention.

What is claimed is:

1. A device for laparoscopic surgery, comprising:
a tip having a main port at a distal end, a proximal port at a proximal end, a lumen extending from the main port to the proximal port, a flexible and non-conductive wall, and an attachment area extending from the proximal end toward the distal end;
wherein the wall includes a preformed shape; and
wherein the tip is removably attached to a rigid cannula of a laparoscopic device, the laparoscopic device being configured to perform suction or irrigation at a surgical site.

2. The device for laparoscopic surgery of claim 1, wherein the attachment area fits around the rigid cannula, and an inner surface of the flexible wall mechanically interacts with the outer surface of the cannula.

3. The device for laparoscopic surgery of claim 1, wherein the attachment area fits inside the lumen of the rigid cannula, and an outer surface of the flexible wall mechanically interacts with the inner surface of the rigid cannula.

4. The device for laparoscopic surgery of claim 1, wherein the preformed shape includes one of the following: a straight shape; a 180 degree curve; a 90 degree curve; a 45 degree curve.

5. The device for laparoscopic surgery of claim 1, wherein the preformed shape includes an extension between a curve and the distal end.

6. The device for laparoscopic surgery of claim 1, further comprising a terminal section extending from the distal end toward the proximal end, wherein the terminal section includes one or more venting ports extending through the wall and configured to allow the transit of fluid and/or debris.

7. The device for laparoscopic surgery of claim 1, further comprising a specialized terminal section that includes one of: a Frazier tip, or a Poole tip.

8. The device for laparoscopic surgery of claim 1, further comprising one or more fiducial markers to aid visibility or placement of the tip during a surgical procedure.

9. The device for laparoscopic surgery of claim 1, wherein the tip is fixedly attached to a rigid cannula, and wherein the rigid cannula is removably attached to the laparoscopic device.

10. A system for laparoscopic surgery, comprising:
a laparoscopic device configured to perform suction or irrigation at a surgical site;
a tip having a main port, a proximal port, a lumen extending from the main port to the proximal port, and a flexible wall, wherein the flexible wall has a shape for facilitating access to the surgical site, and wherein the tip is removably attached to the laparoscopic device; and
one of the following coupled to the laparoscopic device: a vacuum source for supplying a suction force, or a fluid source for supplying a fluid.

11. The system for laparoscopic surgery of claim 10, wherein the tip further comprises one or more venting ports configured to allow the transit of fluid and/or debris.

12. The system for laparoscopic surgery of claim 10, the tip further comprising a specialized terminal section that is located near the main port that includes one of: a Frazier tip, or a Poole tip.

13. The system for laparoscopic surgery of claim 10, the tip further comprising fiducial markers to improve visibility or placement of the tip during a surgical procedure.

14. The system for laparoscopic surgery of claim 10, wherein the system is useable concurrently with one or more laparoscopic instruments.

15. A method of using a device for laparoscopic surgery, comprising:
attaching a flexible tip to a cannula of a laparoscopic device, wherein the tip has a shape and a distal end, and wherein the device is configured to perform suction or irrigation at a surgical site in a body cavity;
passing the device and attached tip through a trocar, wherein the tip flexes to allow passage through the trocar and returns to the shape when in the cavity;
positioning the distal end proximate to the surgical site; and
introducing a fluid to the surgical site using a fluid source coupled to the laparoscopic device.

16. The method of using a device for laparoscopic surgery of claim 15, wherein the introducing step comprises:
introducing a suction force to the surgical site using a vacuum source coupled to the laparoscopic device.

17. The method of using a device for laparoscopic surgery of claim 15, wherein the fluid is one of the following: a saline solution, or a medication.

18. The method of using a device for laparoscopic surgery of claim 16, wherein the suction force is for removing a fluid or debris from the surgical site.

19. The method of using a device for laparoscopic surgery of claim 16, wherein the suction force is for improving visualization of the surgical site.

20. The method of using a device for laparoscopic surgery of claim 15, wherein the device is positioned to allow access to the surgical site by one or more laparoscopic instruments.
